(19) United States
(12) Patent Application Publication
(10) Pub. No.: US 2004/0122334 A1
(43) Pub. Date: Jun. 24, 2004

(54) Vantilation and volume change measurements using permanent magnet and magnet sensor affixed to body

(75) Inventor: Stanley M. Yamashiro, Anaheim, CA (US)

Correspondence Address: McDERMOTT, WILL & EMERY, Professional Corporation, Suite 3400, 2049 Century Park East, Los Angeles, CA, 90067-3208, US

(73) Assignees: Alfred E. Mann Institute for Biomedical Engineering at the University of Southern; California

(21) Appl. No.: 10/664,549

(22) Filed: Sep. 19, 2003

Related U.S. Application Data
(60) Provisional application No. 60/412,129, filed on Sep. 19, 2002.

(51) Int. Cl. A61B 5/08

(52) U.S. Cl. 600/534

(57) ABSTRACT

Apparatus for measuring ventilation of a body having a surface that moves in response to the ventilation. The apparatus may include a first permanent magnet, a first magnet attachment mechanism configured to attach the first magnet to the surface, a first magnetic sensor having an output, a first sensor attachment mechanism configured to attach the first sensor to the surface, and a processing system configured to communicate with the magnetic sensor and to determine the ventilation based on the output of the magnetic sensor. An associated process is also disclosed, along with broader applications of the apparatus and process.

### CROSS-REFERENCE TO RELATED APPLICATIONS

This application is related and claims priority to U.S. provisional application serial No. 60/412,129, filed Sep. 19, 2003, entitled “Non-intrusive Measurement of Ventilation and Body Volume Changes By Magnetic Plethysmography,” the entire content of which is incorporated herein by reference.

### BACKGROUND

1. Field

This application relates to the measurement of ventilation, as well as to the measurement of volume changes in body parts.

2. Related Art

The measurement or detect of ventilation, such as the respiration of a human subject, is often necessary or desirable. Performing this task in a non-intrusive manner and without the use of a mouthpiece or mask is also often necessary or desired.

Efforts have been made to address this need by measuring changes in the volume of the lung and/or the abdomen by monitoring movement of the rib cage and/or abdominal wall. These efforts, however, may not be wholly satisfactory.

Systems that use coils (e.g., U.S. Pat. Nos. 4,815,473 and 4,258,718), for example, can be subject to drift. This can be particularly problematic in applications requiring changes to be measured over a long period, such as during sleep or exercise. These systems can also be subject to interference from stray magnetic fields.

Systems that measure circumferential changes in the chest (e.g., U.S. Pat. No. 4,815,473) can also be subject to artifacts caused by deformation of the soft tissue.

Some of these systems, moreover, can be complex and bulky. To measure both the movement of the chest and abdomen, for example, can require two complete sets of movement detection mechanisms (e.g., U.S. Pat. No. 4,258,718).

The use of permanent magnets has also been proposed (e.g., U.S. Pat. No. 5,825,293). This system, however, can restrict the mobility of the human subject and be subject to errors due to movement of the subject.

### SUMMARY

Apparatus may measure ventilation of a body having a surface that moves in response to the ventilation. The apparatus may include a first permanent magnet, a first magnet attachment mechanism configured to attach the first magnet to the surface, a first magnetic sensor having an output, a first sensor attachment mechanism configured to attach the first sensor to the surface, and a processing system configured to communicate with the magnetic sensor and to determine the ventilation based on the output of the magnetic sensor.

The apparatus may include a second permanent magnet and a second magnet attachment mechanism configured to attach the second magnet to the surface. The strength of the first and second magnets may be different.

The apparatus may include a second magnetic sensor and a second sensor attachment mechanism configured to attach the second sensor to the surface. The sensitivity of the first and second sensors may be different.

The magnet attachment mechanism and the sensor attachment mechanism may include an adhesive.

The magnet attachment mechanism and the sensor attachment mechanism may include tape.

The magnet attachment mechanism and the sensor attachment mechanism may include a strap.

The apparatus may include a first electrode. The magnet attachment mechanism may be configured to attach the first electrode and the magnetic to the surface at substantially the same time. Similarly, the apparatus may include a second electrode, and the sensor attachment mechanism may be configured to attach the second electrode and the sensor to the surface at substantially the same time.

The magnet and the first electrode may be affixed to the magnetic attachment mechanism, and the sensor and the second electrode may be affixed to the sensor attachment mechanism.

The first and second electrodes may be configured to operate with an electrocardiograph.

A process may measure ventilation of a body having a surface that moves in response to the ventilation. The process may include affixing a first permanent magnet to a first location of the surface, affixing a first magnetic sensor having an output to a second location on the surface that is different from the first location, and processing the output of the first sensor to determine the ventilation.

The process may include affixing a second magnetic sensor to a third location of the surface.

The process may include affixing a second permanent magnet to a third location of the surface.

The surface may include a chest, abdomen and back, and the first location may be on the chest, the second location may be on the back, and the third location may be on the abdomen.

The relative strengths between the magnetic fields from the first and second magnets at the location of the sensor may be correlated to the amounts by which the abdomen and chest move during the ventilation.

The process may include an adjustment to achieve the relative strengths.

The adjustment may be to the location of the first or second magnet on the surface.

The adjustment may be to the location of the sensor on the surface.

The adjustment may be to the strength of the first or second magnet.

The first and second magnets may each have a set of poles. These poles may be oriented in substantially the same direction. These poles may be oriented in substantially the opposite direction.

The angular position of the magnet relative to the sensor may be such as to substantially maximize changes in the output from the sensor caused by corresponding changes in the distance between the magnet and the sensor. Alternatively, optimum position may be such as to maximize linearity of the relationship between sensor output as a function of changes in the anterior-posterior or lateral dimension of a subject.

The process may include attaching a first electrode to the surface at substantially the same time as the magnet is attached to the surface, and attaching a second electrode to the surface at substantially the same time as the sensor is attached to the surface.

The first and second electrodes may each have an output and the output of the first electrode and the output of the second electrode may be use to produce an electrocardiogram.

The ventilation may be determined at substantially the same time as the electrocardiogram is produced.

Apparatus may measure ventilation of a body having a surface that moves in response to the ventilation. The apparatus may include a first permanent magnet attached at a first location to the surface, a magnetic sensor attached at a second location to the surface that is different from the first location, and a processing system configured to communicate with the magnetic sensor and to determine the ventilation based on the output of the magnetic sensor.

The apparatus may include a second permanent magnet attached at a third location to the surface that is different from the first and the second location.

The first and second magnets may each have a set of poles. The set of poles may be oriented in substantially the same direction. The set of poles may be oriented in substantially the opposite direction.

Apparatus may measure the change in the volume of a body part having a surface that moves in response to the change. The apparatus may include a permanent magnet, a magnet attachment mechanism configured to attach the magnet to the surface, a magnetic sensor having an output, a sensor attachment mechanism configured to attach the sensor to the surface and a processing system configured to communicate with the magnetic sensor and to determine the change in volume based on the output of the magnetic sensor.

A process may measure the change in the volume of a body part having a surface that moves in response to the change. The process may include affixing a permanent magnet to a first location of the surface, affixing a magnetic sensor having an output to a second location on the surface that is different from the first location, and processing the output of the sensor to determine the ventilation.

A biomedical sensor may be used in connection with a body having a surface. The sensor may include a permanent magnet, an electrode and an attachment mechanism configured to attach the electrode and the magnet to the surface at substantially the same time.

A biomedical sensor may be used in connection with a body having a surface. The sensor may include a magnetic sensor, an electrode and an attachment mechanism configured to attach the electrode and the sensor to the surface at substantially the same time.

These, as well as still further features, objects and benefits will now become clear from a review of the detailed description of illustrative embodiments below and the accompanying drawings.

### BRIEF DESCRIPTION OF DRAWINGS

FIGS. 1(a)-1(g) illustrate a permanent magnet and a magnetic sensor being affixed to different locations on a surface of a body that moves in response to ventilation.

FIGS. 2(a)-2(b) illustrate a set of permanent magnets and a set of magnetic sensors being affixed to different locations on a surface of a body that moves in response to ventilation.

FIGS. 3(a)-3(c) illustrate a set of permanent magnets and a magnetic sensor being affixed to different locations on a surface of a body that moves in response to ventilation.

FIGS. 4(a)-4(c) illustrate location adjustments that may be made to affect the relative strengths between the magnetic fields from a first and a second permanent magnet at the location of a magnetic sensor.

FIGS. 5(a)-5(b) illustrate size adjustments that may be made to affect the relative strengths between the magnetic fields from a first and a second permanent magnet at the location of a magnetic sensor.

FIGS. 6(a)-6(d) illustrate a set of permanent magnets and a magnetic sensor affixed to different locations on a surface of a body that moves in response to ventilation, along with various orientations for the poles of the magnets.

FIG. 7 is a block diagram of a system for measuring ventilation.

FIGS. 8(a)-8(c) illustrate attachment mechanisms for attaching a permanent magnet or magnetic sensor to the surface of a body.

FIG. 9 illustrates a biomedical sensor that attaches a permanent magnet or magnetic sensor to the surface of a body at the same time as an electrode.

### DETAILED DESCRIPTION OF ILLUSTRATIVE EMBODIMENTS

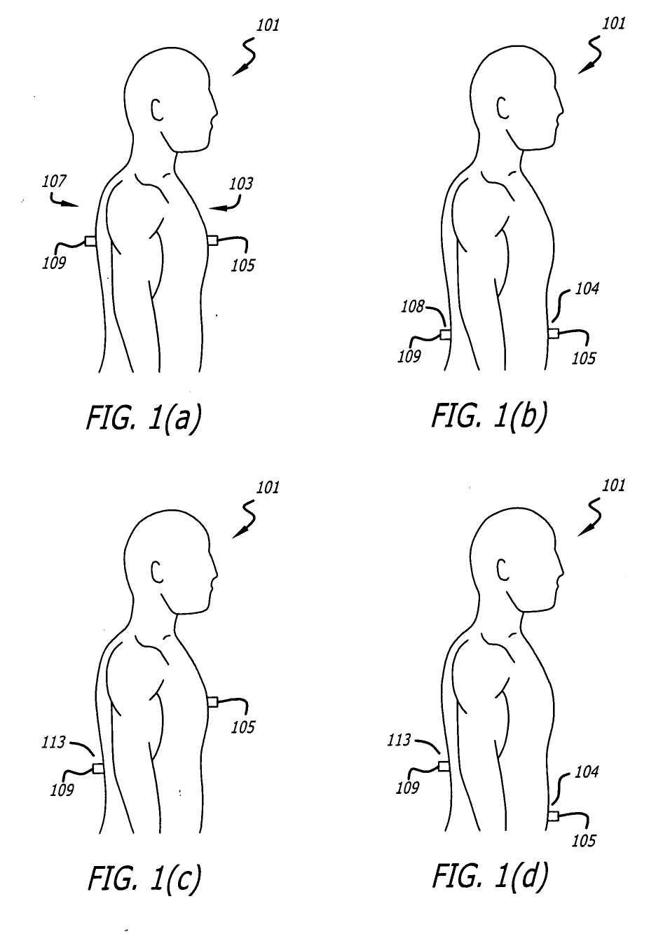

FIGS. 1(a)-1(g) illustrate a permanent magnet and a magnet sensor affixed to different locations on a surface of a body that moves in response to ventilation.

As shown in FIG. 1(a), a human subject 101 has a chest 103 to which a magnet 105 may be affixed. The human subject 101 also has an upper back 107 to which a sensor 109 may be affixed.

As is well known, the chest 103 and upper back 107 are parts of a surface, i.e., the upper torso, that moves in response to ventilation by the subject 101. The chest 103 and the upper back 107 typically move apart from one another during inhalation and toward one another during exhalation.

The magnet 105 may be a permanent magnet. It may be made from the rare earth samarium cobalt or from neodymium. It may also be made from any other type of material or combination of materials.

The sensor 109 may be a magnetic sensor. It may be a magnetoresistive device, including an anisotropic or giant magnetoresistive device. It may be a Hall effect device. The magnetic sensor may sense magnetic fields along only a single axis. It may also be any other type of magnetic sensor or a combination of types of magnetic sensors.

The strength of the magnetic field generated by the magnet 105 and the corresponding sensitivity of the sensor 109 may be of a magnitude that permits the sensor 109 to detect changes in the separation distance between the magnet 105 and the sensor 109 that are caused by ventilation. The strength of the magnetic field generated by the magnet 105 may be sufficient to allow the field from the magnet 105 to be distinguished at the location of the sensor 109 from other environmental fields. Both the strength of the magnetic field from the magnet 105 and the sensitivity of the sensor 109 may remain substantially the same over long periods of time and in varying environmental conditions. Errors commonly attributed to drift can thus be minimized. Drift has long-since made it difficult for current systems to reliably measure changes in end-expiratory lung volume (EELV) level. EELV is known to change during exercise and in response to therapeutic application of positive airway pressure (CPAP). CPAP is the primary therapy used to treat sleep apnea.

The angular position of the magnet 105 relative to the sensor 109 may be such as to substantially maximize the changes in the output from the sensor 109 that are caused by corresponding changes in the distance between the magnet 105 and the sensor 109. This optimum position may be such as to cause the magnetic field that is generated by the magnet 105 to be substantially perpendicular to the sensing axis of the sensor 109. Alternatively, optimum position may be such as to maximize linearity of the relationship between sensor output as a function of changes in the anterior-posterior or lateral dimension of a subject.

The magnet 105 may be attached to the surface of the subject 101 in a broad variety of locations. In FIGS. 1(a) and 1(c), it is shown as being attached to the chest 103 of the human subject 101 near the sternum. The magnet 105 may instead be attached near the umbilicus 104 as shown in FIGS. 1(b) and 1(d), to the upper, lateral rib cage 110 as shown in FIG. 1(e), just below the lateral rib cage 112 as shown in FIG. 1(f) or to the lower lateral rib cage 114 as shown in FIG. 1(g). It may also be placed at other locations.

Similarly, the sensor 109 may be placed at a broad variety of locations. It is shown in FIG. 1(a) as being attached to the upper back 107 over the spine. It may also be attached to the lower back 108 over the spine as shown in FIG. 1(b), to the middle back 113 over the spine as shown in FIGS. 1(c) and 1(d), to the upper, lateral rib cage 110 as shown in FIGS. 1(e) and 1(g) or just below the lateral rib cage 112 as shown in FIG. 1(f). It may also be placed at other locations.

The location of the magnet 105 and the sensor 109 may also be switched in FIGS. 1(a)-8(g). The magnet 105 and sensor 109 may also be located in the transverse plane or in any other plane. They can be directly across from one another, as illustrated in FIGS. 1(a), 1(b), 1(e) and 1(f), or offset from one another, as illustrated I FIGS. 1(c), 1(d) and 1(g).

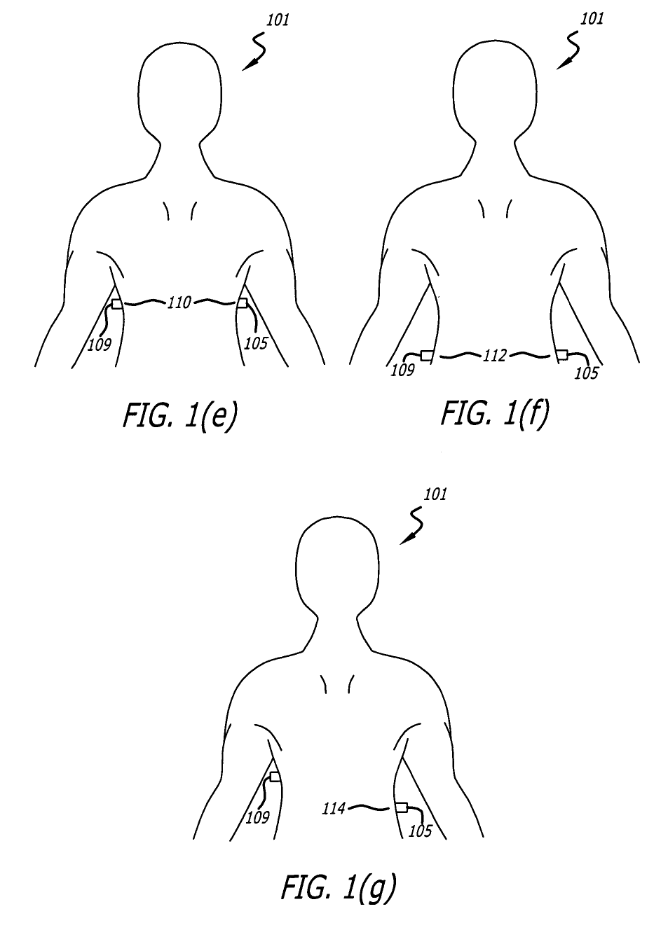

FIGS. 2(a)-2(b) illustrate a set of permanent magnets and a set of magnetic sensors affixed to different locations on the surface of a body that moves in response to ventilation.

As shown in FIG. 2(a), a human subject 201 has a chest 203 to which a magnet 205 may be affixed near the sternum. The subject 201 also has an abdomen 207 to which a second magnet 209 may be affixed near the umbilicus. The subject 201 also has an upper back 211 to which may be affixed a sensor 213 and a lower back 215 to which may be affixed a sensor 217.

The magnets 205 and 209 and the sensors 213 and 217 may be any of the types described above in connection with the magnet 105 and sensor 109 in FIG. 1(a).

In the configuration shown in FIG. 2(a), movement of both the lungs and abdomen may be monitored as an indication of ventilation. The movement of the lung may primarily be detected by the combination of the magnet 205 and the sensor 213; the movement of the abdomen may be primarily be detected by the combination of the magnet 209 and the sensor 217.

As shown in FIG. 2(b), the magnet 205 and the associated sensor 213 may also be positioned on the upper, lateral rib cage 210 while the magnet 209 and the associated sensor 217 may be positioned just below the lateral rib cage 212.

Each magnet/sensor paid may also be placed in other positions, including opposite of the positions shown in FIGS. 2(a) and 2(b).

The output of the sensors 213 and 217 may be representative of ventilation of the subject 201. The amount of the ventilation may be equated to a function of the output of the sensors 213 and 217, such as the sum of the outputs.

One or more of the outputs of the sensors 213 and 217 may be multiplied by a weighting factor to enhance the linearity of the ventilation measurement. The weighting factor may be based on the relative degree to which the abdomen and chest move during ventilation.

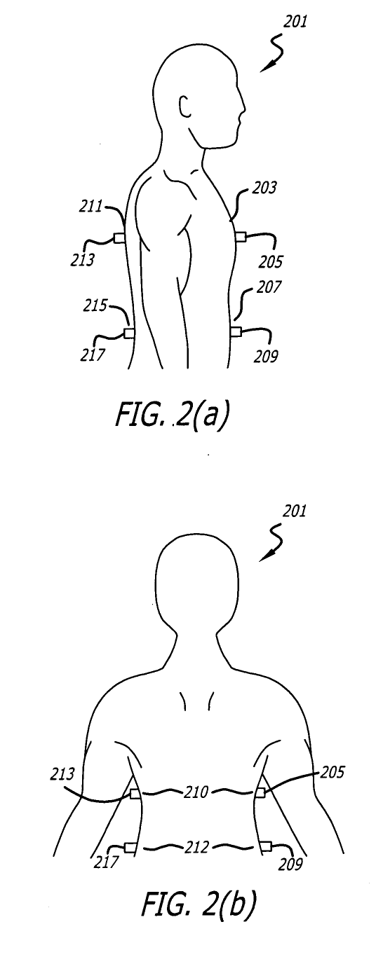

FIGS. 3(a)-3(c) illustrate a set of permanent magnets and a magnetic sensor affixed to different locations on the surface of a body that moves in response to ventilation. As shown in FIG. 3(a), a human subject 301 may have a magnet 303 affixed to his chest 305 near his sternum and a magnet 307 affixed to his abdomen 309 near his umbilicus. A sensor 311 may be attached to the middle back 310 of the subject 301 over the spine.

The magnets 303 and 307 and the sensor 311 may be any of the types discussed above in connection with the magnet 105 and the sensor 109 in FIG. 1(a).

The fields from the magnets 303 and 307 may be superimposed at the location of the sensor 311. The output of the sensor 311 may therefore be the sum of the field strengths from the magnets 303 and 307 at the location of the sensor 311. The contributions made to the indication of ventilation by the lungs and abdomen of the subject 301 may thus be added together at the sensor 311, thus eliminating the need for having a separate sensor for each magnet.

The locations of the magnets 303 and 307 and the sensor 311 can vary widely. FIG. 3(b) illustrates another configuration in which the magnets 303 and 307 may be affixed to the upper back 312 and the lower back 314 of the subject 301 over the spine, respectively, while the sensor 311 may be affixed just below the sternum 313.

FIG. 3(c) illustrates a still further configuration in which the magnets 303 and 307 are attached to the upper, lateral rib cage 316 and just below the lateral rib cage 318, respectively, and the sensor 311 is attached to the lower, lateral rib cage 320.

Although having thus-far discussed FIGS. 3(a)-3(c) in the context of having two magnets and one sensor, a similar result can be achieved by having two sensors and one magnet. In this situation, each magnet in FIGS. 3(a)-3(c) may be replaced by a sensor, and each sensor may be replaced by a magnet. In this configuration, the output of the two sensors may be combined electronically to generate a single signal that is representative of ventilation.

The signal generated by the sensor 311 may not vary linearly with the ventilation. One cause of this may be that the lung and abdomen expand in differently in response to the same ventilation, but that the corresponding magnetic fields from the lung monitor (magnet 303) and the abdomen monitor (magnet 307) vary at the location of the sensor in the same amounts.

To compensate for this, a weighting adjustment of the system may be made. A broad variety of types of adjustment can be made.

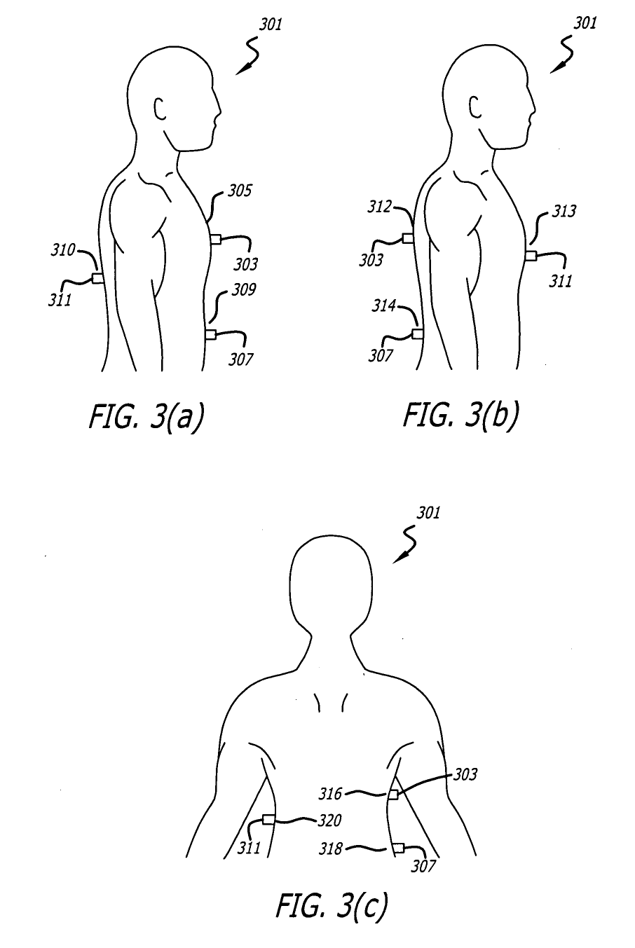

FIGS. 4(a)-4(d) illustrate location adjustments that may be made to affect the relative strengths between the magnetic fields from a first and a second permanent magnet at the location of a magnetic sensor.

As shown in FIGS. 4(a)-4(c), a human subject 401 may have affixed to his chest 403 near his sternum a magnet 405. He may also have affixed near his umbilicus 404 a magnet 407. He may also have affixed in approximately the center of his back 408 near his spine a sensor 409. The magnets 405 and 407 and the sensor 409 may be any of the types described above in connection with the magnet 105 and the sensor 109 in FIG. 1(a).

In order to adjust the weighting of the magnetic fields at the location of the sensor 409 from the magnets 405 and 407, the magnet 407 may be moved and attached to a different location on the subject 401. The movement may be upward, as shown in FIG. 4(a), downward, laterally, or in another direction.

FIG. 4(b) illustrates an adjustment approach in connection with the same magnets and sensor, except that the magnet 405 is moved instead. The movement may be downward, as shown in FIG. 4(b), upward, laterally or in another direction.

FIG. 4(c) illustrates another adjustment approach, this time moving the sensor 409. The sensor 409 may be moved upwardly as shown in FIG. 4(c), downwardly, laterally or in another direction.

The magnets and sensors shown in FIGS. 4(a)-4(c) may also be at other locations, such as those discussed above in connection with FIGS. 3(a)-3(c). Each magnet may be replaced by a sensor, and the sensor may be replaced by a magnet.

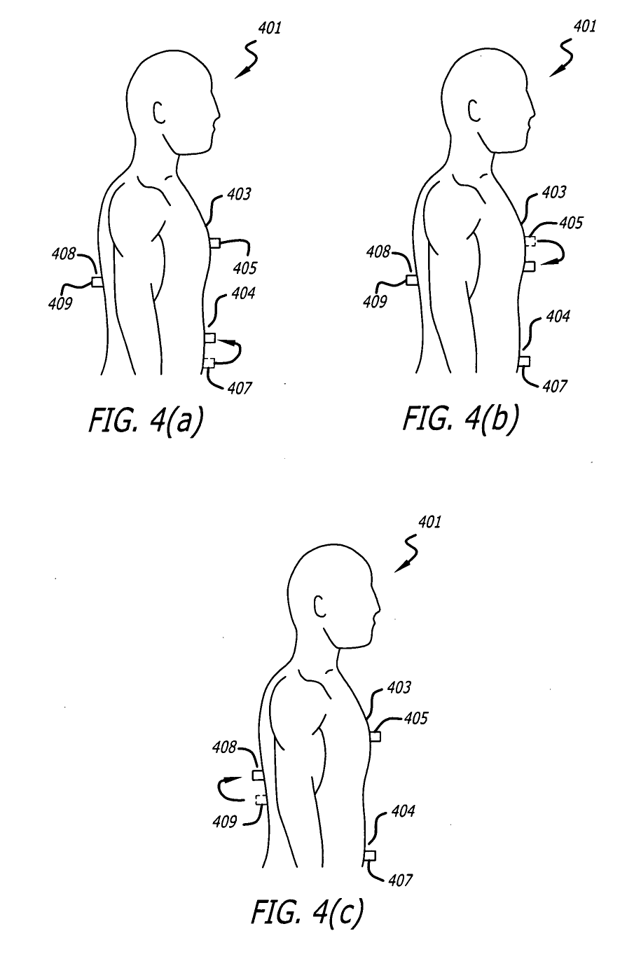

FIGS. 5(a)-5(b) illustrate size adjustments that may be made to affect the relative strengths between the magnetic fields from a first and a second permanent magnet at the location of a magnetic sensor. As shown in FIG. 5(a), a subject 501 may have attached to his chest 503 near his sternum a magnet 505. He may also have attached near his umbilicus 504 a magnet 507. He may also have attached a sensor 509 in approximately the middle of his back 508 near his spine.

Another approach for adjusting the relative magnitude of the field strengths at the sensor 509 from the magnets 505 and 507 is to vary the size of one of the magnets, such as to increase the size of the magnet 505, as shown in FIG. 5(a), or to increase the size of the magnet 507, as shown in FIG. 5(b). Decreases in size may also be made. The magnets 505 and 507 and the sensor 509 may be in any of the locations discussed above in connections with FIGS. 3(a)-3(c), and a sensor may be substituted for each magnet and a magnet substituted for the sensor.

Another approach for adjusting the relative field strengths of the magnetic fields from the magnets at the location of the sensor is to vary the number of magnets at each location. Their fields may or may not be aligned.

Other weighting approaches may also be used. For example, multiple sensors may be used in connection with a single magnet, and the weighting may be done in the electronics that are associated with the multiple sensors. The alignment of the poles of one or more of the magnets or the alignment of the sensing axis of the sensor may also be adjusted. One or more of the adjustment techniques that have now been described, or other adjustment techniques, may be combined.

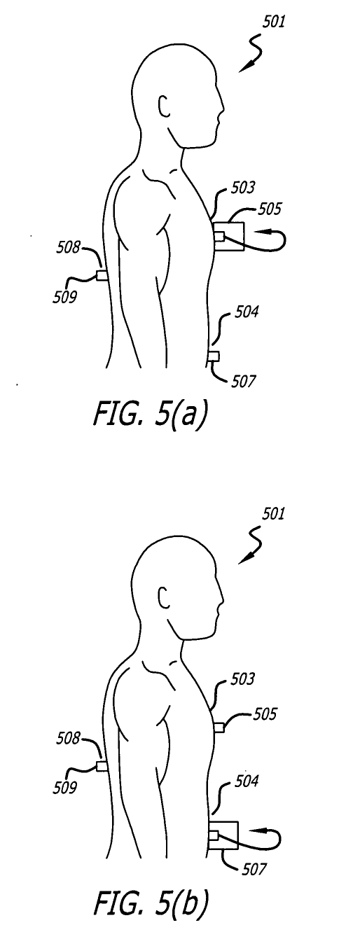

FIGS. 6(a)-6(d) illustrate a set of permanent magnets and a magnetic sensor affixed to different locations on the surface of a body that move in response to ventilation, along with various orientations for the poles of the magnets.

As shown in FIGS. 6(a) and 6(b), a subject 601 may have attached to his chest 603 near his sternum a magnet 605. He may also have attached a magnet 609 to his abdomen 607 near his umbilicus. He may also have attached a sensor 611 to approximately the middle of his back 610. The magnets 605 and 609 and the sensor 611 may be any of the types discussed above in connection with the magnet 105 and the sensor 109 shown in FIG. 1(a).

The poles of the magnets 605 and 609 may both be vertically oriented in the same direction, as shown in FIG. 6(a); horizontally oriented in the same direction, as shown in FIG. 6(c); vertically oriented in opposite directions, as shown in FIG. 6(b); or horizontally oriented in opposite directions, as shown in FIG. 6(d). Different pole orientations may also be used. The magnets and the sensor may also be in other locations, as discussed above in connection with FIGS. 3(a)-3(c). Each magnet may also be replaced by a sensor, and the sensor may be replaced by a magnet. In this latter case, the sensing axis of each sensor may be aligned in the same or opposite direction or in other relative directions.

Alignment of the poles in the same direction may cause the magnetic fields generated by the magnets to be added at the location of the sensor, while alignment of the poles in opposite directions may cause those fields to be subtracted at the location of the sensor. Different applications may benefit from different alignments.

For example, orienting the magnetic poles as shown in FIG. 6(d) may lead to a net vertical magnetic field at the sensor location which varies substantially as a linear function of the volume of ventilation. Weighting, as discussed above, may also be used to enhance this linearity.

Orienting the poles as shown in FIG. 6(c) may cause the configuration to effectively detect obstructions to breathing, such as may occur during sleep apnea.

Although having thus-far discussed only two magnets in a configuration, a different number of magnets could be used instead, such as one, three, four or five. When using more than a single magnet, the multiple magnets may be attached to the subject in the same area of the body, or in different areas. The multiple magnets may all be sensed by a single sensor or by multiple sensors.

Similarly, two, three or more sensors may be used, placed at the same location or at different locations. They may be used in connection with a single magnet or multiple magnets.

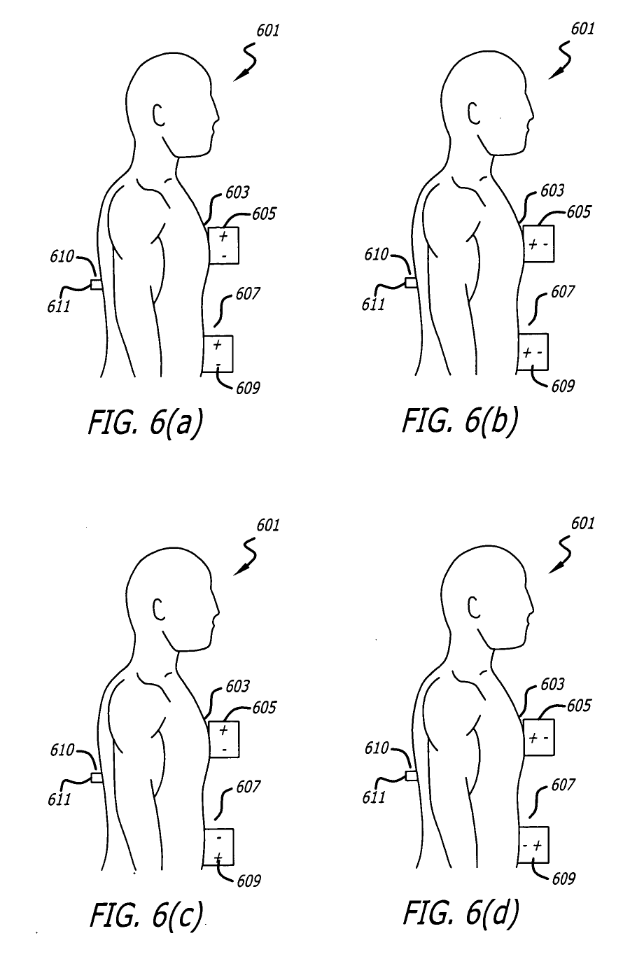

FIG. 7 is a block diagram of a system for measuring ventilation. As shown in FIG. 7, one or more magnets 701 may radiate a magnetic field to a sensor 703. The magnets 701 and the sensor 703 may be any of the types discussed above in connection with the magnet 105 and the sensor 109 in FIG. 1(a), and may be positioned and attached to body of a subject in any of the positions and in any of the numbers discussed above in connection with FIGS. 1(a)-1(g), 2(a)-2(b) and 3(a)-3(c). They may also be weighted in any of the fashions discussed above in connection with FIGS. 4(a)-4(c) and 5(a)-5(b). Their poles and/or sensing axes may also be in any of the orientations discussed above in connection with FIGS. 6(a)-6(d).

The output of the sensor 703 may be in communication with a processing system 705, which may be in communication with an output device 707.

The communication between the sensor 703 and the processing system 705 may be over a wired or a wireless connection. The processing system 705 may be location in close proximity to the sensor 703 or remote from the sensor 703.

Similarly, the communication between the processing system 705 and the output device 707 may be wired or wireless and the output device 707 may be at the same location as the processing system 705 or may be located remotely from it.

In one application, the processing system 705 and/or the output device 707 may be located in a different room from the sensor 703, thus allowing the subject, such as an infant or an elderly person, to be monitored from a different room.

The communication between the sensor 703 and the processing system 705 and/or the processing system 705 and the output device 707 may be over a network system, such as the Internet, thereby allowing remote monitoring and/or diagnosis.

A wireless connection may also be advantageous when monitoring a patient in an ambulance or when monitoring a person in water for the purpose of guarding against drowning.

The output device 707 may be a display, a data storage device, a communications device, a sound device, any other type of device, or any combination of these. It may communicate and/or record the volume of ventilation as a function of time, whether the ventilation exceeds or fails to meet a specified threshold, and/or the results of any type of data analysis that may be done on the output of the sensor 703, such as a determination that ventilation has ceased and/or sleep apnea.

The processing system 705 may be configured to receive the signal from the sensor 703 (or the signals from multiple sensors, if such a configuration is used) and to generate the necessary output for the output device 707. The processing system 705 may include a general purpose computer or may be a system that is dedicated to a ventilation application. It may include hardware, software or combinations of both.

The processing system 705 may be configured to compensate for nonlinearities in the signal from the sensor 703. Various techniques may be used to provide this compensation, including filtering techniques, computational techniques and/or mapping techniques.

The processing system 705 may also include one or more filters configured to eliminate interference and/or errors caused by extraneous magnetic fields. For example, the processing system 705 may include a low pass filter that substantially reduces frequencies above those being generated by breathing, such as above 20 Hz.

The processing system 705 may be contained on an integrated circuit, have very low power requirements, and/or be operated by a battery.

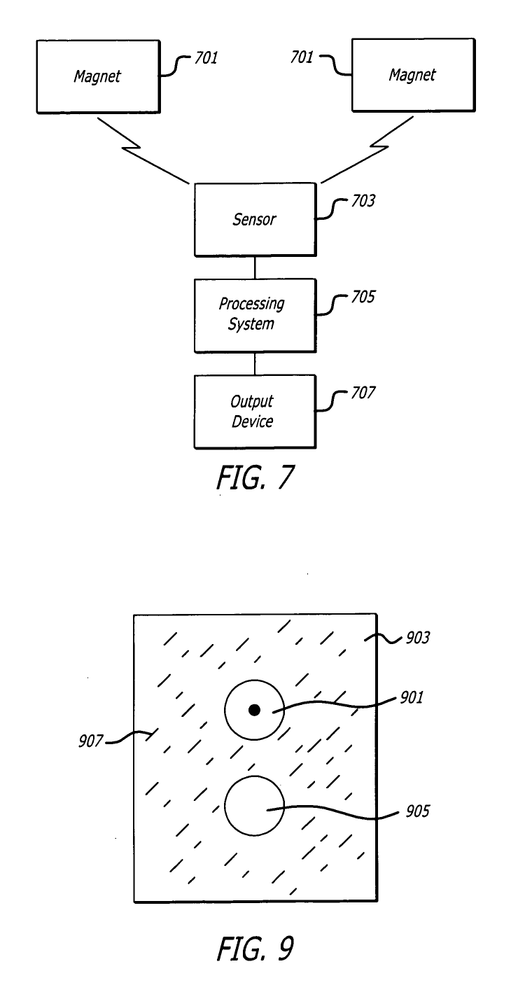

FIGS. 8(a)-8(c) illustrate attachment mechanisms for attaching a permanent magnet or a magnetic sensor to the surface of a body. Any one of these, as well as any other approach, may be used to effectuate any of the attachments discussed above in connection with FIGS. 1-6.

FIG. 8(a) illustrates a magnet or sensor 801 being attached to a surface 803 of a body using an adhesive 805. The adhesive 805 may be attached to the magnet or sensor 801 at the time that the magnet or sensor 801 is attached to the surface 803. The adhesive 805 may instead be attached to the magnet or sensor 801 well before, in which case the exposed surface of the adhesive 805 may be protected by a non-stick, peel-off cover.

FIG. 8(b) illustrates another embodiment of an attachment mechanism. As shown in FIG. 8(b), the magnet or sensor 801 may be attached to the surface of the body 803 using a length of adhesive tape 807. The adhesive tape 807 may contain adhesive 809 that adheres both to the magnet or sensor 801 and to the surface of the body 803.

FIG. 8(c) illustrates another attachment mechanism. As shown in FIG. 8(c), the magnet or sensor 801 is attached to the surface 803 of the body using a belt 805 wrapped around the entire circumference of the subject 807. The belt 805 may have a detachable buckle whose position on the belt may be adjusted to accommodate for different circumferences. The belt 805 may also include elastic material in addition to or instead of the adjustable buckles for this purpose.

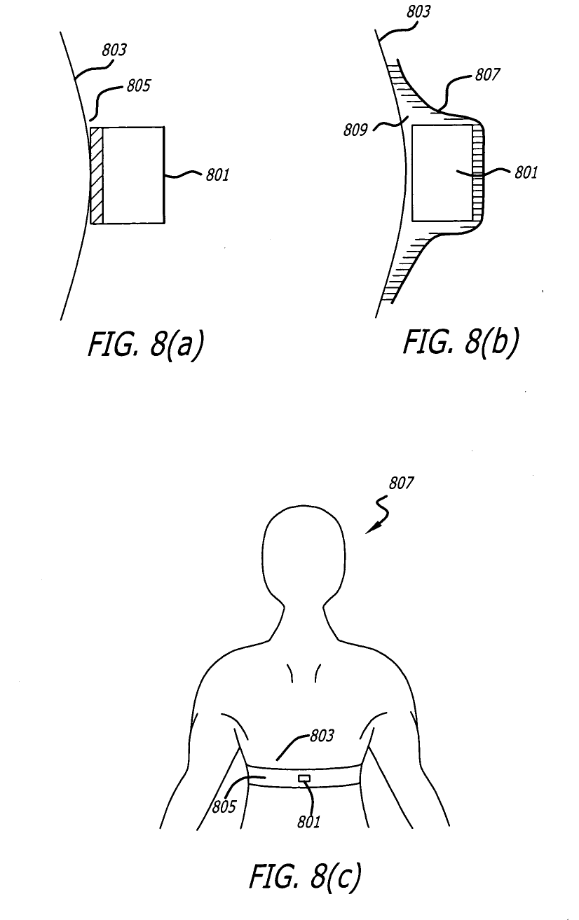

FIG. 9 illustrates a biomedical sensor that attaches a permanent magnet or magnet sensor to the surface of a body at the same time as an electrode. As shown in FIG. 9, a magnet or sensor 901 is attached to a pad 903. The pad 903 may also have attached to it an electrode 905. Adhesive material 907 may also be included on the pad 903 to facilitate attaching the pad with the magnet 901 and the electrode 905 to the surface of the body of a subject (not shown in FIG. 9).

The electrode 905 may be attached by a wire (or wirelessly) to an electrocardiograph, thus assisting in the production of an electrocardiogram. Typically, an electrocardiograph utilizes three connections to the body: a positive connection, a negative connection and ground. The configuration shown in FIG. 9 may be produced in triplicate, with an electrode being attached to each pad, a magnet being attached to one of the pads, a magnet or sensor being attached to the second pad, and a sensor being attached to the third pad. With this configuration, the two magnets (or one sensor) and one sensor (or two magnets) may be affixed to the subject for the purpose of configuring a ventilation system of the type discussed above in connection with FIGS. 3(a)-3(c), FIGS. 6(a)-6(d) and FIG. 7, while simultaneously attaching the three electrodes necessary for generating an electrocardiogram without any additional effort. Both respiration and cardiac activity may then be monitored simultaneously.

Other attachment mechanisms could be used instead to simultaneously attach a magnet or sensor and an electrode to the surface of a subject.

The magnet and sensor configurations and systems that have been discussed above may be used in determining volume changes of any body part, not merely the lungs and/or abdomen. For example, the system may be used in connection with vascular edema and in measuring muscle girth changes during contraction.

What has now been set forth are merely examples of the broad array of components, configurations, methods and steps that may be used and the attributes and benefits that may be obtained. The scope of this application is limited solely by the claims that now follow and their equivalents.

I claim:

1. Apparatus for measuring ventilation of a body having a surface that moves in response to the ventilation comprising:
a first permanent magnet;
a first magnet attachment mechanism configured to attach the first magnet to the surface;
a first magnetic sensor having an output;
a first sensor attachment mechanism configured to attach the first sensor to the surface; and
a processing system configured to communicate with the magnetic sensor and to determine the ventilation based on the output of the magnetic sensor.

2. The apparatus of claim 1 further including:
a second permanent magnet; and
a second magnet attachment mechanism configured to attach the second magnet to the surface.

3. The apparatus of claim 2 wherein the strength of the first and second magnets are different.

4. The apparatus of claim 1 further including:
a second magnetic sensor; and
a second sensor attachment mechanism configured to attach the second sensor to the surface.

5. The apparatus of claim 4 wherein the sensitivity of the first and second sensors are different.

6. The apparatus of claim 1 wherein the magnet attachment mechanism and the sensor attachment mechanism include an adhesive.

7. The apparatus of claim 1 wherein the magnet attachment mechanism and the sensor attachment mechanism include tape.

8. The apparatus of claim 1 wherein the magnet attachment mechanism and the sensor attachment mechanism include a strap.

9. The apparatus of claim 1 further including:
a first electrode wherein the magnet attachment mechanism is also configured to attach the first electrode and the magnetic to the surface at substantially the same time; and
a second electrode wherein the sensor attachment mechanism is configured to attach the second electrode and the sensor to the surface at substantially the same time.

10. The apparatus of claim 9 wherein the first and second electrodes are configured to operate with an electrocardiograph.

11. The apparatus of claim 9 wherein:
the magnet and the first electrode are affixed to the magnetic attachment mechanism; and
the sensor and the second electrode are affixed to the sensor attachment mechanism.

12. A process for measuring ventilation of a body having a surface that moves in response to the ventilation comprising:
affixing a first permanent magnet to a first location of the surface;
affixing a first magnetic sensor having an output to a second location on the surface that is different from the first location; and
processing the output of the first sensor to determine the ventilation.

13. The process of claim 12 further including affixing a second magnetic sensor to a third location of the surface.

14. The process of claim 12 further including affixing a second permanent magnet to a third location of the surface.

15. The process of claim 14 wherein the surface includes a chest, abdomen and back and wherein the first location is on the chest, the second location is on the back, and the third location is on the abdomen.

16. The process of claim 15 wherein the relative strengths between the magnetic fields from the first and second magnets at the location of the sensor are correlated to the amounts by which the abdomen and chest move during the ventilation.

17. The process of claim 16 further including an adjustment to achieve the relative strengths.

18. The process of claim 17 wherein the adjustment is to the location of the first or second magnet on the surface.

19. The process of claim 17 wherein the adjustment is to the location of the sensor on the surface.

20. The process of claim 17 wherein the adjustment is to strength of the first or second magnet.

21. The process of claim 14 wherein the first and second magnets each have a set of poles and wherein the sets of poles are oriented in substantially the same direction.

22. The process of claim 14 wherein the first and second magnets each have a set of poles and wherein the sets of poles are oriented in substantially the opposite direction.

23. The process of claim 12 wherein the angular position of the magnet relative to the sensor is such as to substantially maximize changes in the output from the sensor caused by corresponding changes in the distance between the magnet and the sensor.

24. The process of claim 12 further including:
attaching a first electrode to the surface at substantially the same time as the magnet is attached to the surface; and
attaching a second electrode to the surface at substantially the same time as the sensor is attached to the surface.

25. The process of claim 24 wherein the first and second electrodes each have an output and further including processing the output of the first electrode and the output of the second electrode as part of the production of an electrocardiogram.

26. The process of claim 25 wherein the ventilation is determined at substantially the same time as the electrocardiogram is produced.

27. Apparatus for measuring ventilation of a body having a surface that moves in response to the ventilation comprising:
a first permanent magnet attached at a first location to the surface;
a magnetic sensor attached at a second location to the surface that is different from the first; and
a processing system configured to communicate with the magnetic sensor and to determine the ventilation based on the output of the magnetic sensor.

28. The apparatus of claim 27 further including a second permanent magnet attached at a third location to the surface that is different from the first and the second location.

29. The apparatus of claim 28 wherein the first and second magnets each have a set of poles and wherein the set of poles are oriented in substantially the same direction.

30. The apparatus of claim 29 wherein the first and second magnets each have a set of poles and wherein the set of poles are oriented in substantially the opposite direction.

31. Apparatus for measuring the change in the volume of a body part having a surface that moves in response to the change comprising:
a permanent magnet;
a magnet attachment mechanism configured to attach the magnet to the surface;
a magnetic sensor having an output;
a sensor attachment mechanism configured to attach the sensor to the surface; and
a processing system configured to communicate with the magnetic sensor and to determine the change in volume based on the output of the magnetic sensor.

32. A process for measuring the change in the volume of a body part having a surface that moves in response to the change comprising:
affixing a permanent magnet to a first location of the surface;
affixing a magnetic sensor having an output to a second location on the surface that is different from the first location; and
processing the output of the sensor to determine the ventilation.

33. A biomedical sensor for use in connection with a body having a surface comprising:
a permanent magnet;
an electrode; and
an attachment mechanism configured to attach the electrode and the magnet to the surface at substantially the same time.

34. A biomedical sensor for use in connection with a body having a surface comprising:
a magnetic sensor;
an electrode; and
an attachment mechanism configured to attach the electrode and the sensor to a first area of the surface at substantially the same time.

35. A biomedical sensor for use in connection with a body having a surface comprising:
a first permanent magnet;
a first electrode;
a first attachment mechanism configured to attach the first electrode and the first magnet to the surface at substantially the same time;
a first magnetic sensor;
a second electrode;
a second attachment mechanism configured to attach the second electrode and the first sensor to a second area of the surface at substantially the same time;
a second permanent magnet or second magnetic sensor;
a third electrode; and
a third attachment mechanism configured to attach the third electrode and the second magnet or the second sensor to a third area of the surface at substantially the same time.
